(19) United States
(12) Patent Application Publication
(10) Pub. No.: US 2022/0362438 A1
(43) Pub. Date: Nov. 17, 2022

(54) Derivatized or Rapidly Polymerizing Collagen Compositions for Tissue Augmentation Containing Nonresorbable or Slowly Resorbable Polymers

(71) Applicant: Shanghai Qisheng Biological Preparation Co., Ltd., Shanghai (CN)

(72) Inventors: Dale P. DeVore, Chelmsford, MA (US); Jiaxun ZHU, Shanghai (CN); Lixia JIANG, Shanghai (CN); Peng DU, Shanghai (CN); Changzheng WEI, Shanghai (CN); Jianying WU, Shanghai (CN)

(21) Appl. No.: 17/744,248

(22) Filed: May 13, 2022

Related U.S. Application Data
(60) Provisional application No. 63/188,261, filed on May 13, 2021.

(51) Int. Cl. A61L 27/26; A61L 27/34; A61L 27/54

(52) U.S. Cl. CPC A61L 27/26; A61L 27/34; A61L 27/54; A61L 2300/622; A61L 2400/06; A61L 2430/34

(57) ABSTRACT

Proived herein are derivatized or rapidly polymerizing collagen compositions for tissue augmentation containing non-resorbable or slowly resorbable polymers. Also provided are methods for the preparation of the compositions, and methods for augmenting soft tissue utilizing the compositions.

### CROSS REFERENCE TO RELATED APPLICATION

This application claims the benefit of U.S. Provisional App. No. 63/188,261 filed May 13, 2021, which is incorporated by reference in its entirety for all purposes.

### FIELD OF INVENTION

The present invention describes compositions for augmenting soft tissue using injectable, derivatized collagen-based formulations or in situ polymerizable collagen gels. The soluble derivatized collagen compositions or in situ polymerizable collagen gels may contain collagen fibers, polymethylmethacrylate (PMMA) spheres, or slow resorbable biospheres composed of polyethylene glycol (PEG), or spheres of polylactide (PLA) and polyglycolide (PLG) polymers and copolymers thereof (PLGA), or granules composed of other polymers such as L-Lactide/Trimethyl carbonate (Lactoprene®) or calcium hydroxyapatite spheres or poly ϵ-caprolactone (PCL) spheres or poly(p-dioxanone) (PDO) spheres.

The derivatized collagen compositions or in situ polymerizable collagen gels provide varying degrees of in vivo longevity dependent on the stability of the fibrous collagen and/or the biodegradation profiles of the spherical particles, granules or nanoparticles.

### BACKGROUND

Use of bioabsorbable materials for facial soft tissue augmentation dates back to the early 1980s when bovine collagen was introduced to treat lines, wrinkles and volume defects. Since then, a variety of non-permanent, absorbable dermal fillers and facial implants have been approved and used worldwide (hyaluronic acid, collagen and porcine small intestinal submucosa). Semi-permanent and permanent dermal fillers have also been developed. Semi-permanent materials include hydroxylapatite and poly L-lactic acid. Non-absorbable materials such as PMMA microspheres and PTFE facial implant strands have also been used to correct facial defects.

Collagen-based compositions are well known dermal fillers and have been thoroughly reviewed by Cockerham and Hsu (Facial Plastic Surgery, 25:106-113, 2009) and Denton and Shoman (Office Based Cosmetic Procedures & Technology, Section 3, pp 59-64, 2010). Injectable collagen dermal fillers include bovine-based Zyderm and Zyplast, human cell-derived collagen, Cosmoderm and Cosmoplast, human tissue derived Autologen, Dermalogen, and Cymetra, porcine-based Evolence and most recently rapidly polymerizing collagen gels that appear clear and transparent and rapidly form collagen fibers after implantation into tissues (see U.S. Pat. No. 10,111,981). These collagen-based dermal filler products contain injectable collagen fibrils.

One permanent dermal filler, Artefill (now Bellafill) contains PMMA spheres combined with a denatured collagen carrier. There are several literature publications describing this product. (add references). Derma Veil (Tagle et. al.), is a product combining glycolic acid and polylactic acid. Together, polylactic acid and glycolic acid act in concert to 1) stimulate collagen production and 2) hydrate the outer layers of the skin (Tagle et al.). This combination does not include a collagen carrier.

The present invention describes the application of chemically derivatized collagen or rapidly polymerizing collagen gels as a carrier for intact collagen fibrils/fibers, PMMA, PEGs, and PLGA spheres and particles.

While, the preparation of chemically derivatized collagen compositions have been described by Miyata (U.S. Pat. No. 4,164,559) and DeVore, et al (U.S. Pat. Nos. 4,713,446, 4,851,513, 4,969,912, 5,067,961, 5,104,957, 5,201,764, 5,219,895, 5,332,809, 5,354,336, 5,476,515, 5,480,427, 5,631,243, and 6,161,544), none teaches the application of derivatized collagen compositions for use as dermal fillers or as carriers for spheres and particles PMMA, PEG, PLG and similar synthetic compositions. The preparation of rapidly polyermizing collagen gels has been described in U.S. Pat. No. 10,111,981.

### SUMMARY OF INVENTION

This invention relates to an injectable, chemically derivatized collagen solution or rapidly polymerizing collagen gels containing collagen fibers, non-bioresorbable PMMA microspheres (32-50 μm diameter), or slowly resorbable PGA or L-lactide/D-lactide or L-lactide/glycolide copolymers-PLGA (20-50 μm diameter) or calcium hydroxyapatite spheres or poly ϵ-caprolactone (PCL) spheres or poly(p-dioxanone) (PDO) spheres for soft tissue augmentation and tissue regeneration.

The chemically derivatized collagen or rapidly polymerizing collagen gels may be prepared from bovine, porcine, or human collagen, including recombinant human collagen solution.

The compositions are injectable through 25-30 gauge needles into dermal and subdermal tissues to replenish or correct deficient dermal tissue such as those associated with wrinkles and folds.

### BRIEF DESCRTPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1: Light absorbance change during fibrillogenesis of soluble collagen solutions in Example 2.

FIG. 2: SDS-PAGE of glutaric anhydride modified collagen (GA-collagen) showed a increasing molecular weight in all bands. (Lane 1: marker, Lane2: 3 mg/mL bovine atelocollagen standard, Lanes 3 & 4: procine atelocollagen, Lane 5: GA-collagen).

FIG. 3: appearances of glutaric anhydride modified collagen (GA-collagen, left), rapid polymerizing collagen (RPC, middle) and 1:1 (v:v) mixure of GA-collagen and RPC (GA-collagen+RPC, right).

FIG. 4: fibrillogenesis of RPC (up) and GA-collagen+RPC (down) in vitro showing the fibrous units of the combination are thicker than RPC alone.

FIG. 5: In vivo fibrillogenesis of RPC (20% fibrillar collagen in weight) in rabbit ear: TEM of RPC implanted in rabbit ear for 5 minute (center cut), D band showed self-assembly of collagen molecules.

### DETAILED DESCRIPTION OF THE INVENTION

The following description and examples illustrate embodiments of the invention in detail. It is to be understood that this invention is not limited to the particular embodiments described herein and as such can vary. Those of skill in the art will recognize that there are numerous variations and modifications of this invention, which are encompassed within its scope.

All patents, patent applications, and literature references are hereby incorporated by reference in their entirety.

The present invention provides a biologically compatible collagenous reaction product with plastic properties created by incorporating ethylenically unsaturated or polymeric substituents into collagen. The substituents are incorporated by reacting suitable collagen with an acylating agent containing ethylene moieties or acylating group rich polymers. The resulting collagen solution is supplemented with nondegradable PMMA spheres or slowly degradable spheres, such as those composed of polyethylene glycol or copolymers of lactides and glycolides.

Also used herein is a rapidly polymerizing collagen (RPC) gel, such as that described in U.S. Pat. No. 10,111,981B2. The RPC may comprise a neutralized solution comprising an acid soluble collagen, EDTA and a polyol, and wherein the acid soluble collagen comprises collagen selected from the group consisting of Type I collagen, Type III collagen and combinations thereof.

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described.

As used herein, the term “a” or “an” is intended to mean “one or more” (i.e., at least one) of the grammatical object of the article. Singular expressions, unless defined otherwise in contexts, include plural expressions. By way of example, “an element” means one element or more than one element.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

The use of “or” means “and/or” unless stated otherwise.

As used herein, unless otherwise noted, the term “comprise”, “include” and “including” will be understood to imply the inclusion of a stated step or element or group of steps or elements but not the exclusion of any other step or element or group of steps or elements.

The phrase “consisting of” is meant to include, and is limited to, whatever follows the phrase “consisting of.” Thus, the phrase “consisting of” indicates that the listed elements are required or mandatory and that other elements may be present.

As employed herein, the term “biologically compatible” refers to collagen modified in accordance with the present invention (i.e., a collagenous reaction product) or collagen compositions formulated in accordance with the present invention which is stable when incorporated or implanted into or placed adjacent to the biological tissue of a subject and more particularly, does not deteriorate appreciably over time or induce an immune response or deleterious tissue reaction after such incorporation or implantation or placement.

As defined herein, the term “injectable collagen composition” refers to an injectable, chemically modified, biologically compatible collagen composition and such compositions supplemented with nonbioresorbable or resorbable microspheres which when injected into tissue, augments deficient tissue, such as skin lines and folds. The term “biologically compatible” refers to collagen compositions formulated in accordance with the present invention which when incorporated or implanted into or placed adjacent to the biological tissue of a subject, does not deteriorate appreciably over time or induce an immune response or deleterious tissue reaction after such incorporation or implantation or placement.

DeVore et al. (U.S. Pat. Nos. 4,713,446 and 4,851,513) and Miyata et al. (U.S. Pat. No. 4,748,152) have developed collagen-based viscoelastic solutions wherein the collagen is derivatized at lysyl amino acids to increase its solubility at neutral pH. DeVore, et al (U.S. Pat. No. 10,111,981) describes the preparation and general application of collagen gels that instantaneously form collagen fiber units following implantation into tissues.

DeVore et al. (supra) teaches a method of producing collagen suitable for viscoelastic solutions by acylating the lysyl amino acids of collagen with a combination of monofunctional and bifunctional reagents. The result of this acylation treatment is that some collagen lysine amino groups are modified to substitute a carboxylic acid group in place of the basic amino function. The residue of the lysine amino groups are covalently linked to lysine amino groups of the same or an adjacent collagen molecule. The viscoelastic solution is produced by reconstituting this derivatized collagen in a physiologic saline solution.

A preferred acylating reaction for the solubilization of collagen is taught in U.S. Pat. Nos. 4,851,513 and 4,713,446. Also preferred is acylation using glutaric anhydride (reaction pH between about 7.0 and about 9.0).

An effective amount of the acylating agent will vary within limits but generally comprises from about 0.5 to about 20 weight percent total collagen, preferably from about 5 to about 10 weight percent total collagen in solution. The effective amount of the acylating agent will be based on the total amount of collagen in the solution.

Acylation of the collagen is carried out at alkaline pH, for example, in the range of from about 8.0 to about 10.0 pH, preferably at about pH 9.0 or so. In order to achieve complete acylation of the collagen that is being treated, which is desirable because it leads to better performance in the final shaped implant articles (i.e., the properties of the incorporated monomers are better imparted to the final articles), the collagen should be filtered and solubilized. Using conventional filtering means, e.g., a millipore filter with a 3 μm pore size, the collagen can be filtered to remove impurities and contaminants. The filtered collagen can then be solubilized (i.e., dissolved or dispersed) in a suitable proteolytic solution, e.g., pepsin.

It has also been found that the reaction between the collagen and the acylating agent may require more than one reaction “run.” That is, additional acylating agent can be added to the initial reaction mixture (i.e., the initial collagen and the initial acylating agent) to continue the reaction to completion, i.e., complete acylation of the collagen being treated.

The reaction time for the acylation of the collagen will vary according to a number of factors including the amount of collagen to be acylated, the type of acylating agent, the pH and temperature of the reaction mixture, to name just a few factors. In addition, the method of addition of the acylating agent to the suitable collagen will affect the reaction time. For example, addition of the acylating agent as a solid or in an appropriate solution will increase and decrease the reaction time, respectively. Reaction time is generally longer if the acylating agents are added as solids or powders.

In general, the acylation reaction should proceed to completion within a time ranging from about 5 to about 90 minutes, preferably from about 20 to about 40 minutes. The acylation reaction should generally be carried out at a temperature of from about 4.degree. to about 37.degree. C., preferably from about 4.degree. to 25.degree. C.

The acylation reaction can be monitored by the decrease in pH. The reaction is complete when pH is stable at 9.0. The reaction can also be monitored by removing aliquots and measuring the free amine concentration of precipitated, washed collagen product.

The reaction can be stopped by adjusting the pH to 12.0 for 2 minutes which destroys the acylating agents. The modified collagen is then precipitated by reducing the pH using hydrochloric acid, acetic acid, nitric acid, sulfuric acid, or other acid.

The amount of acid added should be sufficient to cause the pH of the reaction mixture to fall to below pH 5.0, preferably from about pH 4.0 to about 4.5 or so. When the acid is added to the mixture, and it is suggested that the addition be in small quantities, e.g., dropwise, the mixture should become cloudy indicating a change to acidic pH of the collagen mixture as the modified or reacted collagen “falls out of solution.”

The biological stability of the chemically modified collagen composition of the invention may be affected by the solubility characteristics of the starting collagen as well as the extent of chemical modification. Completely solubilized modified collagen generally does not produce a composition that is resistant to high concentrations of neutral proteolytic enzymes under laboratory testing conditions. Hence, in practicing this invention, prior to chemical modification, a solubilized collagen solution is converted to partially fibrillar collagen. Chemical modification of a partially fibrillar collagen solution results in a modified collagen composition which is clear, transparent and injectable. The use of partially fibrillized collagen as the preferred starting material for the modification process results in an injectable composition with improved resistance to degradation by neutral proteolytic enzymes, such as trypsin.

To prepare a partially fibrillized collagen solution, a solubilized collagen solution is adjusted to pH between about 7.0 and 7.6, preferably about 7.4, and allowed to undergo limited fibrillogenesis at a temperature between about 25. degree. C. and 40. degree. C., preferably about 37. degree. C. , for a period of between about 10 and 30 minutes, preferably about 20 minutes.

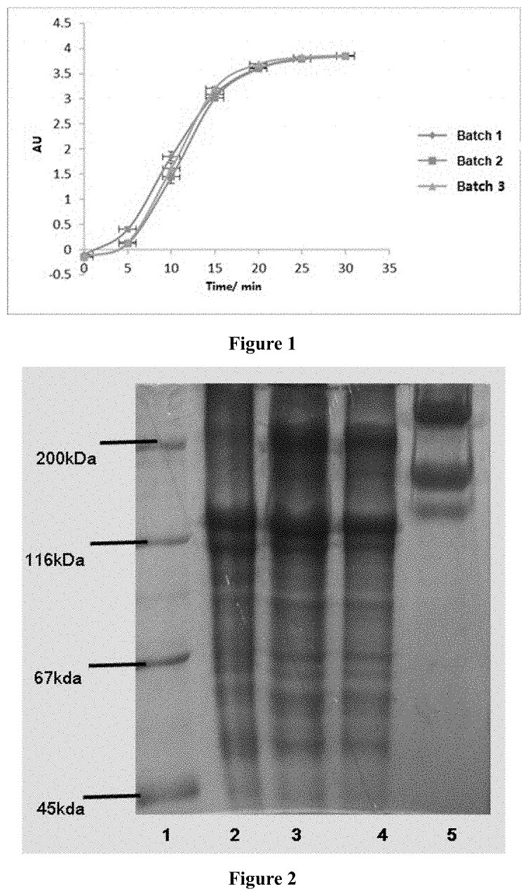

The extent of fibrillogenesis can be ascertained by measuring the increase in turbidity or light absorption of the solubilized collagen solution by absorption spectroscopy (for example, as shown in FIG. 1). In general, fibrillogenesis is permitted to continue until the turbidity of the solution becomes about 20% to 60% greater, preferably about 25% greater, than the absorbance of the initial solution.

After chemical modification, the partially fibrillar collagen composition loses its turbidity and turns clear and transparent. Collagen microfibrils in the chemically modified partially fibrillar collagen solutions can no longer be observed microscopically. The modified partially fibrillar collagen solution is believed to include modified collagen molecules and modified collagen aggregates containing collagen molecules.

Acylation reactions have been used to derivatize soluble and insoluble collagen and have been described by DeVore, et. al. in a series of patents (U.S. Pat. Nos. 4,713,446; 4,851,513; 4,969,912; 5,067,961; 5,104,957; 5,201,764; 5,219,895; 5,332,809; 5,354,336; 5,476,515; 5,480,427; 5,631,243; and 6,161,544). However, none of these patents describe the use of chemically derivatized collagen combined with non-resorbable or slowly resorbable substances, such as PMMA spheres, PLG spheres or particles, or other non-resorbable or slowly resorbable particles or spheres to treat soft tissue deficiencies or defects.

In the present invention, acylation reactions have been used to derivatize soluble collagen with agents that react with deprotonated free amines, particularly on lysine amino acids. Sulfonic acids, anhydrides, sulfonyl chlorides, and acid chlorides are classes of chemical compounds that react with free amines of proteins resulting in the covalent attachment of the specific chemical moieties to proteins. These compounds are commonly known as acylation reagents.

Specific acylation agents have been used to alter the net charge and charge density of proteins. Certain agents can be used to change the net charge from positive to negative.

Certain agents can change the net charge from one positive to two negatives per reacted site. Specific agents include, but are not limited to, 3,5-dicarboxybenzenesulfonyl chloride and others.

Suitable acylating agents for use in the instant invention include aliphatic, acyclic and aromatic anhydrides or acid halides. Non-limiting examples of acylating agents include glutaric anhydride, succinic anhydride, lauric anhydride, diglycolic anhydride, methylsuccinic anhydride, methyl glutaric anhydride, dimethyl glutaric anhydride, succinyl chloride, glutaryl chloride, lauryl chloride, phthalic anhydride, methacrylic anhydride, trifluoroacetic anhydride, styrene/maleic anhydride co-polymer, and ethylene/maleic anhydride copolymer. These chemicals are available from such as Aldrich Chemical Company (Milwaukee, Wis.). Preferred acylating agent for use in the present invention are glutaryl anhydride, methacrylic anhydride, trifluoroacetic anhydride, ethylene/maleic anhydride copolymer, and phthalic anhydride. An effective amount of an acylating agent is broadly between about 0.5 and 20% wt total collagen, preferably between about 3 and 10% total collagen in solution.

Useful sulfonating agents for use as co-acylation agents include but not limited to aliphatic, acyclic and aromatic sulfonic acids or sulfonyl halides. Non-limiting examples of sulfonating agents for use in the present invention include anthraquinone-1,5-disulfonic acid, 2-(chlorosulfonyl)-anthraquinone, 8-hydroxyquinoline sulfonic acid, 2-naphthalene-sulfonyl chloride, beta-styrene sulfonyl chloride, 2-acrylamido-2-methyl-1-propane sulfonic acid, aniline sulfonic acid, fluorosulfonylbenzene sulfonyl chloride, and poly(vinyl) sulfonic acid. These chemicals are also available from such as Aldrich Chemical Company (Milwaukee, Wis.). An effective amount of sulfonating agent is broadly between about 0.5 and 20 wt % of the total collagen, preferably between about 1 and 10 wt % of the total collagen in solution.

Non-limiting combinations of acylating agents and/or sulfonating agents include glutaric anhydride/beta-styrene sulfonyl chloride/methacrylic anhydride; glutaric anhydride/ethylene/maleic anhydride copolymer/methacrylic anhydride; glutaric anhydride/polyvinyl sulfonic acid/methacrylic anhydride; and glutaric anhydride/ethylene/maleic anhydride copolymer/styrene/maleic anhydride copolymer. Preferred combinations for use in the present invention are glutaric anhydride/beta-styrene sulfonyl chloride; glutaric anhydride/phthalic anhydride; and glutaric anhydride/aniline-2-sulfonic acid.

When combinations of two or more acylating agents, sulfonating agents, or mixtures of both agents are used for preparation of modified collagen composition, the total amount of chemical modifiers is preferably between about 3 and 10% wt of collagen in solution. Excess quantities of chemical modifiers beyond the preferred range may result in a collagen composition that is biologically unstable and sensitive to tissue proteases.

Modification of collagen is carried out at alkaline pH, in a range between about 7.5 and 10.0, preferably between about 8.5 and 9.5, and most preferably at about pH 9.0. The acylation reaction can be monitored by the decrease in pH. The reaction is terminated when the pH value remains stable at between about 5 and 8, preferably about 6.5 and 7.5. The reaction can also be monitored by removing aliquots and measuring the free amine concentration of the modified collagen solution as compared to the starting solution of collagen.

The modification reaction should be complete in between about 5 and 90 minutes, preferably between about 20 and 40 minutes. The reactions should be carried out at temperatures between about 0. degree. C. and 37. degree. C., preferably between about 4. degree. C. and 25. degree. C.

The reaction can be stopped by adjusting the pH to about 12.0 for about 2 minutes. This destroys residual, unreacted chemical modifiers. The modified collagen is then precipitated by reducing the pH using hydrochloric acid, acetic acid, nitric acid, sulfuric acid, or other acid.

The amount of acid must be sufficient to precipitate out the chemically modified collagen. Generally precipitation occurs at a pH between about 3.5 and 6.0, preferably between about 4.0 and 5.0.

The precipitate of reacted collagen which now contains substituent groups reacted with amine groups (primarily epsilon-amino groups), is recovered from the mixture using conventional techniques such as centrifugation or filtration. Centrifugation at between about 3,000 and 15,000 rpm for between about 20 and 60 minutes, preferably between about 4,000 and 12,000, for between about 20 and 30 minutes provides efficient recovery of the precipitate.

After recovery, the precipitate is washed with deionized water and subsequently dissolved in a physiological solution, e.g., phosphate buffer (0.1M) at about pH 7.2. It may be necessary to adjust the pH to between about 7.0 and 7.5. This can be done, for example, by the addition of sodium hydroxide solution.

Following dissolution of the precipitate, the solution is generally filtered by conventional filtering means, for example a 5 micron filter, and then centrifuged to remove air bubbles. At this point, the resulting solution containing chemically modified collagen molecules and aggregates exhibits a viscous consistency, varying degrees of transparency and clarity. The viscosity of the injectable modified collagen solution, determined at a temperature of about 25.degree. C., is broadly between about 30,000 centipoise and 300,000 centipoise, preferably between about 75,000 and 150,000 centipoise. Viscosity of the solution may be adjusted by the addition of buffer or collagen precipitate.

The extent of modification may be modulated by varying the amount of chemical modifiers, the pH, the temperature and the time of the reaction. In addition, the method of addition of the modifying agents will affect the reaction. Reactions are generally slower if the chemical agent is added as a solid or powder rather than as a solution.

The extent of modification also determines the biological stability of the collagen-based composition. Complete modification results in a collagen solution that rapidly degrades in the presence of neutral proteolytic enzymes, such as trypsin. It has been discovered that the biological stability of collagen composition can be manipulated by controlling the extent of chemical modification.

The modified collagen solutions are pseudoplastic as such solutions exhibit shear thinning as shear rate increases, typical of pseudoplastic solutions. They are also thixotropic as such solutions regain their original viscosity after thinning due to shear such as injection through a syringe.

The chemically modified collagen compositions can be injected into superficial dermis, mid-dermis, or deep dermis to correct contour defects in facial skin or such compositions can be injected into the loose connective tissue surrounding lip muscle or into the body of the lip to enhance lip appearance. The collagen compositions are injectable through a 30 gauge needle. The material remains colorless and provides a long-lasting clinical effect. The collagen compositions can be prepackaged in ready-to-use syringes containing materials exhibiting several different degrees of durability

To prepare derivatized collagen compositions with extended durability, the collagen solutions are supplemented with collagen fibers, 10-30% PMMA spheres (25-50 μm diameter), 10-30% PEG spheres or particles having a diameter of approximately 25-50 μm) or 10-30% spheres or particles of lactide/lactide or lactide/glycolide having similar dimensions. Derivatized collagen solutions containing collagen fibers provide a soft tissue filler with durability similar to previous collagen products, PMMA supplemented compositions provide a soft tissue filler with permanent durability. PEG and lactide/glycolide compositions (PLGA) provide a soft dermal filler with extended durability compared to non-supplemented derivatized collagen solutions or derivatized collagen solutions containing fibrous collagen units.

Despite derivatized collagents, rapidly polymerizing collagen (RPC) gels can also be used in the composition of the present invention. As used herein, the term “rapidly polymerizing collagen gel” refers to an injectable acid soluble collagen composition comprising, a neutralized solution comprising the acid soluble collagen, EDTA and a polyol, wherein the composition is injectable at neutralized pH, and the acid soluble collagen polymerizes upon exposure to ion-containing fluids. In some embodiments, the rapidly polymerizing collagen gels are as described in U.S. Pat. No. 10,111,981B2.

In some embodiments, the acid soluble collagen comprises collagen selected from the group consisting of Type I collagen, Type III collagen and combinations thereof. In some embodiments, the RPC gel comprises acid soluble collagen in a concentration between 5 and 70 mg/ml, preferably between 25 and 65 mg/ml, more preferably between 20 and 40 mg/ml.

In some embodiments, EDTA comprised in the RPC gel is a disodium EDTA. In some embodiments, the concentration of the EDTA in the RPC gel is between about 10 mM and about 50 mM, preferably between about 25 mM and about 40 mM, more preferably between about 30 mM and 35 mM.

In some embodiments, polyol comprised in the RPC gel is a sugar alcohol, such as D-mannitol. In some embodiments, the concentration of the polyol in the RPC gel is between about 2.5% and 4% (w/v), preferably between about 3.0% and 3.9% (w/v). In some embodiments, the RPC gel further comprises disaccharide, fructose, or combinations thereof.

In some embodiments, the concentration of the the RPC gel has an osmolality of 280-360 mmol/kg.

In some embodiments, when injecting into a soft tissue deficiency, the RPC gel quickly forms an opaque collagen fibril matrix. In some embodiments, the soft tissue deficiency is selected from the group consisting of wrinkles, dermal folds, dermal laxity, skin contour defects, dermal fine lines, dermal furrows and dermal unevenness. In some embodiments, the soft tissue deficiency is in lips or facial skin. In some embodiments, the RPC gel forms a collagen fibril matrix that fills the soft tissue deficiency.

In some embodiments, the RPC gel forms a collagen fibril matrix after exposure to the tissue and the matrix remains durable for at least 4 weeks, preferably at least 12 weeks, more preferably at least 6 months after said injection.

In some embodiments, the injection of the RPC gel stimulates regeneration of the tissue. In some embodiments, the injected RPC gel integrates with matrices of the tissue.

To prepare collagen compositions with extended durability, the RPC collagen solutions are supplemented with collagen fibers, 10-30% PMMA spheres (25-50 μm diameter), 10-30% PEG spheres or particles having a diameter of approximately 25-50 μm) or 10-30% spheres or particles of lactide/lactide or lactide/glycolide having similar dimensions. RPC solutions containing collagen fibers provide a soft tissue filler with durability similar to previous collagen products, PMMA supplemented compositions provide a soft tissue filler with permanent durability. PEG and lactide/glycolide compositions (PLGA) provide a soft dermal filler with extended durability compared to non-supplemented derivatized collagen solutions or derivatized collagen solutions containing fibrous collagen units.

The following enumerated embodiments are representative of the invention:
1. A composition for application in soft tissue augmentation comprising
   (i) derivatized collagen solutions or rapidly polymerizing collagen gels (before undergoing fibrillogenesis) or crosslinked or uncrosslinked collagen fibrils; and
   (ii) non-resorbable or slowly resorbable particles, spheres or granules contained in part (i).
2. The composition of item 1, wherein the source of collagen for part (i) is selected from allogenetic, mammal hides or marine species or axolotl hides derived matrix; and/or
   the collagen is selected from full collagen or atelocollagen, or recombinant collagen or recombinant collagen peptides from microorganism, plants, insect cells or animal cells, or collagen mimic peptides.
3. The composition of item 1, wherein the derivatized collagen is derivatized with acetylation agents that alter the pKa of collagen and has one or more of the following features:
   (a) soluble at neutral pH (such as 6.5-7.5);
   (b) does not undergo fibrillogenesis at physiological pH; and/or
   (c) precipitates at acidic pH (such as 3.5-5.5, preferred 4.0˜5.0).
4. The composition of item 1, wherein the derivatized collagen is derivatized with one or more agents selected from the group consisting of glutaric anhydride, succinic anhydride, maleic anhydride, citric acid anhydride, oxalic acid anhydride and ethylenediamine tetraacetic anhydride.
5. The composition of item 1, wherein the rapidly polymerizing collagen gels are as described in U.S. Pat. No. 10,111,981B2; and/or
   the rapidly polymerizing collagen gels comprises a neutralized solution comprising an acid soluble collagen, EDTA and a polyol, and wherein the acid soluble collagen comprises collagen selected from the group consisting of Type I collagen, Type III collagen and combinations thereof.
6. The composition of item 5, wherein the acid soluble collagen in a concentration between 5 and 70 mg/ml; and/or
   wherein said EDTA is disodium EDTA; and/or
   wherein said EDTA is in a concentration between 10 and 50 mM; and/or
   wherein said polyol is a sugar alcohol, such as D-mannitol; and/or
   wherein said polyol is in a concentration between 2.5% and 4% (w/v); and/or
   wherein said rapidly polymerizing collagen gels further comprises a disaccharide, fructose, or combinations thereof; and/or
   wherein said rapidly polymerizing collagen gel has an osmolality of 280-360 mmol/kg.
7. The composition of item 1, wherein the crosslinked collagen is crosslinked by one or more of the chemical agents selected from the group selected from: aldehyde,such as methyl aldehyde, oxalaldehyde, glutaraldehyde and butenoic aldehyde; or iridoids such as genipin; or carbodiimide such as dicyclohexyl carbodiimide; or epoxide such as 1,4-butanediol diglycidyl ether (BDDGE), or acyl azide; or saccharides such as ribose or glucose.
8. The composition of item 1, wherein the non-resorbable or slowly resorbable particles, spheres or granules are one or more selected from the group consisting of:
   reconstituted or crosslinked collagen fibrils;
   Polymethylmethacrylate (PMMA) microspheres;
   polymethylmethacrylate-hydroxyapatite microspheres;
   crosslinked hyaluronic acid microspheres produced by emulsified crosslinking reaction, double emulsion evaporation method, microfluidic crosslinking reaction, or stamp formation;
   polyethylane glyco (PEG) microspheres;
   PEG-hydroxyapatite microspheres;
   poly-L-lactide (PLA) microspheres;
   PEG-PLA copolymer microspheres;
   poly-L-lactide-hydroxyapatite microspheres;
   polyglycolic acid (PGA) microspheres;
   poly-L-lactide-hydroxyapatite microspheres;
   polylactide and polyglycolide polymers and copolymers (PLGA) microspheres;
   poly ϵ-caprolactone (PCL) microspheres;
   PCL-PLA copolymer microspheres;
   poly-ϵ-caprolactone-hydroxyapatite microspheres;
   poly(p-dioxanone) (PDO) microspheres;
   poly(p-dioxanone)-hydroxyapatite microspheres;
   calcium hydroxyapatite; and
   L-Lactide/Trimethylene Carbonate Copolymer granules.
9. The composition of item 8, wherein the crosslinker of hyaluronic acid micrspheres is selected from divinylsulfone, glutaraldehyde, 1,4-butanediol diglycidyl ether, p-phenylene biscarbodiimide, 1,2,7,8-diepoxyoctane or oligomers rich in amino groups (such as poly-lysine or poly-arginine or γ-polyglutamic acid).
10. The composition of item 8, wherein the crosslinked hyaluronic acid microspheres is coated with bio-degradable polymers, such as poly-L-lactide (PLA), polyethylene glycol (PEG), or PLGA, or poly(p-dioxanone) (PDO).
11. The composition of item 1, wherein the size of the non-resorbable or slowly resorbable particles, spheres or granules is ranged from 5 to 150 μm, preferably from 20 to 50 μm; and/or
   wherein the non-resorbable or slowly resorbable particles, spheres or granules are obtained through spray-precipitation technique, emulsion, double emulsion evaporation method, microfluidic reaction, Solid-Gel process, melt extrusion technique, sintering process or stamp formation; and/or
   wherein the non-resorbable or slowly resorbable particles, spheres or granules are sterilized through heat moist sterilization, gamma irradiation or ethylene oxide sterilization.
12. The composition of item 1, wherein the amount of collagen in part (i) is from 0.1 wt % to 10 wt %, and the amount of part (ii) is from 1 wt % to 55 wt %, based on the total weight of the composition.
13. The composition of item 1, further comprising additive(s) in an amount of from 0 to 5 wt %, based on the total weight of the composition.
14. The composition of item 1, wherein the additive is selected from the group consisting of
   local anesthesia drugs such as lidocaine, procaine, preferably in a concentration of from 0.1% to 0.5% by weight; and/or
   polyols stabilizers, such as glycerin, mannitol, butanediol, sorbitol, preferably in a concentration of from 0.1 to 5% weight; and/or
   a stabilizer with chelating ability, such as EDTA, EGTA, citric acid, sodium citrate, preferably in a concentration of from 0.1 to 5% by weight; and/or
   a sulfur stablizer or dissolution promotor, such as Chondroitin Sulfate Sodium (CS), Gluscosamine Sulphate (GS) or Methyl sulfonyl methane (MSM), preferably in a concentration of from 0.1% to 5% by weight; and/or
   soluble small molecules added through dialysis process, ultrafitration or tangential flow ultrafiltration with organic membranes or ceramic membrane with MWCO>10 KDa.

15. The composition of item 1, wherein part (ii) is added to part (i) by utilizing vacuum planetary mixer to form an injectable homogeneous gel, preferably with a revolution speed of 200 rpm˜1,400 rpm and an autorotation speed of 100 rpm˜700rpm, preferably with a mixing time of 10˜30 minutes with vacuum.
16. The composition of item 1, wherein part (ii) is added to a salt or salt or pH precipitate of part (i) and re-solublized by dialysis or ultradialysis or ultrafiltration process to form a homogeneous injectable gel.
17. A method for the preparation of the composition of any one or items 1˜16, comprising: combining part (i) with part (ii), for example by
   adding part (ii) to part (i) by utilizing vacuum planetary mixer to form an injectable homogeneous gel, preferably with a revolution speed of 200 rpm˜1,400 rpm and an autorotation speed of 100 rpm˜700 rpm, preferably with a mixing time of 10˜30 minutes with vacuum; and/or
   adding part (ii) to a salt or salt or pH precipitate of part (i) and re-solublized by dialysis or ultradialysis or ultrafiltration process to form a homogeneous injectable gel.
18. A method for augmenting soft tissue in a subject in need thereof, comprising injecting the composition of any of items 1-16 to the site in need of the augment.
19. The method of item 18, wherein the composition is injected into soft tissue to correct soft tissue deficiencies; and/or
   wherein the composition is injected into dermis to correct soft tissue deficiencies including wrinkles, dermal folds, dermal laxity, unevenness, facial emaciation, fat atrophy, cheek depression, eye socket depression, or a combination thereof; and/or
   wherein the composition is injected into tissues other than dermis, including cartilage, to correct tissue deficiencies.
20. The method of item 14 wherein the composition is injectable through a 25˜30 gauge needle or cannula, such as a 25, 27 or 30 gauge needle or cannula.

The examples set forth below are intended to illustrate the invention without limiting its scope.

Publications cited herein and the materials for which they are cited are hereby specifically incorporated by reference in their entireties. All reagents, unless otherwise indicated, were obtained commercially. All parts and percentages are by weight unless stated otherwise. An average of results is presented unless otherwise stated. The abbreviations used herein are conventional, unless otherwise defined.

### EXAMPLES

### Example 1

### Preparation of Glutaric Anhydride Modified Collagen

200 mL of 3 mg/mL purified, soluble collagen (Porcogen, Lot #531131080) was filtered through 0.45 μm and 0.2 μm cartridge filters. The filtered collagen was place in a 500 mL beaker and adjusted to a pH of 9.0 using 10N and 1N NaOH. After stirring for 5 minutes at room temperature, pulverized glutaric anhydride powder (Sigma, >95%) was slowly added to the stirring collagen solution at a concentration equal to 10% of the collagen (60 mg). The pH of the collagen solution was maintained at pH 9.0 by addition of drops of 10N NaOH. The glutaric anhydride reaction continued for 15 minutes at which point drops of 6N HCl and 1N HCl were added to reduce the pH to approximately 4.5 to precipitate the derivatized collagen.

The derivatized collagen was then placed in 50 mL centrifuge tubes and centrifuged at 3,500-5,000 rpm to precipitate the derivatized collagen. The recovered precipitate was then solubilized by adjusting the pH to 7.2 by adding drops of 10 N NaOH and 1N NaOH. The pH was monitored as NaOH was mixed with the derivatized collagen pellet. The neutralized, clear and transparent collagen gel was then placed in 50 mL centrifuge tubes and centrifuged to remove air bubbles. SDS-PAGE showed an increasing molecular weigh in both α1, α2 molecule and dimer as well (FIG. 2). The clear, deaerated collagen gel was stored at 2-10° C. until supplemented with collagen fibrils, PMMA spheres, PEG spheres and particles, or lactide/glycolide spheres.

### Example 2

### Preparation and Addition of Collagen Fibrils

Preparation of collagen fibrils. 300 mL of purified, soluble collagen (Porcogen Lot #531131080) was filtered through 0.45 μm and 0.2 μm cartridge filters. The collagen solution was placed in prewashed dialysis tubing (FisherBrand, MW cut-off 12,000-14,000) and placed in a 1L bottle containing phosphate buffer (0.1 M Na₂HPO₄, 0.05 M citric acid, pH7.2) in the presence of NaCl (final concentration was adjusted to 0.15 M). Dialysis was continued for 24 hours to form an opaque fibrous mass. The fibrillar collagen was removed from the dialysis tubing and placed in 50 mL centrifuge tubes. The fibrillar collagen was centrifuged at 3,500 rpm for 15 minutes to concentrate the collagen fibrils. Concentration was estimated by determining dry weight.

Fibrillar collagen was mixed (20% in weight) into derivatized collagen gels and then centrifuged at low speed to remove air bubbles.

Usually Fibrillar collagen induce unfibrillar collagen fibrillogenesis under neutral pH, however, glutaric anhydride modified collagen remains clear, transparent and fluidic with addition of fibirillar collagen.

Collagen molecules can self-assemble into ordered supramolecular structure, which is closely related to the inherent three-strand spiral structure, unique viscous end, molecular chirality and intermolecular interaction force hydrogen bond, electrostatic interaction, hydrophobic interaction. Collagen concentration, temperature, ionic strength and pH are the main factors affecting the collagen fibrillogenesis. Once fibrillogenesis (self-assmbly) is triggered in the proper condition (pH=7.2-7.4 and with 0.1˜0.2M NaCl to provide ionic strenghth), as shown in FIG. 1, self-assembly of collagen molecule automatically spread throughout the solution, causing orderly and stable material appearance and phase change.

### Example 3

### Addition of PMMA Spheres

1. PMMA microspheres (Phosphorex) with diameters from 32-50 μm were mixed into the derivatized collagen solution at 20% by weight of the collagen gel. The microspheres were homogeneously mixed into the collagen gel using two syringes attached by a female-female syringe adaptor. Samples of the PMMA-collagen gel composition were examined microscopically showing a dense concentration of microspheres in the collagen gel. Injectability was determined by extrusion through 27 and 30 gauge needles. Samples were placed in 1.0 mL syringes for subsequent testing in the rabbit ear model.

2. PMMA microspheres (Phosphorex) with diameters from 32-50 μm were mixed into the derivatized collagen solution at 10% by weight of the collagen gel. The microspheres were homogeneously mixed into the collagen gel using two syringes attached by a female-female syringe adaptor. Samples of the PMMA-collagen gel composition were examined microscopically showing a dense concentration of microspheres in the collagen gel. Injectability was determined by extrusion through 27 and 30 gauge needles. Samples were placed in 1.0 mL syringes for subsequent testing in the rabbit ear model.

### Example 4

### Addition of PLGA Microspheres

PLGA microspheres (Phosphorex) with diameters from 20-40 μm are mixed into the derivatized collagen solution at 20% by weight of the collagen gel. The microspheres are homogeneously mixed into the collagen gel using two syringes attached by a female-female syringe adaptor. Samples of the PLGA-collagen gel composition were examined microscopically showing a dense concentration of microspheres in the collagen gel. Injectability was determined by extrusion through 27 and 30 gauge needles. Samples wer placed in 1.0 mL syringes for subsequent testing in the rabbit ear model.

### Example 5

### Addition of Polyethylene Glycol (PEG) Microspheres

Polyethylene glycol microspheres (LifePearl®) with diameters from 45-105 μm are mixed into the derivatized collagen solution at 20% by weight of the collagen gel. The microspheres are homogeneously mixed into the collagen gel using two syringes attached by a female-female syringe adaptor. Samples of the PEG-collagen gel composition were examined microscopically showing a dense concentration of microspheres in the collagen gel. Injectability was determined by extrusion through 27 and 30 gauge needles. Samples were placed in 1.0 mL syringes for subsequent testing in the rabbit ear model.

### Example 6

### Addition of L-Lactide/Trimethylene Carbonate Copolymer Granules

L-Lactide/Trimethylene Carbonate Copolymer granules (Lactoprene® 8812 Polymer) with diameters from 20-40 μm are mixed into the derivatized collagen solution at 20% by weight of the collagen gel. The granules are homogeneously mixed into the collagen gel using two syringes attached by a female-female syringe adaptor. Samples of the L-Lactide/Trimethylene Carbonate Copolymer-collagen gel composition were examined microscopically showing a dense concentration of microspheres in the collagen gel. Injectability was determined by extrusion through 27 and 30 gauge needles. Samples were placed in 1.0 mL syringes for subsequent testing in the rabbit ear model.

### Example 7

### Addition of Calcium Hydroxyapatite Spheres

Calcium hydroxyapatite spheres (from Merz Biomaterials) with diameters from 25-45 μm are mixed into the derivatized collagen solution at 20% by weight of the collagen gel. The granules are homogeneously mixed into the collagen gel using two syringes attached by a female-female syringe adaptor. Samples of the Calcium hydroxyapatite-collagen gel composition were examined microscopically showing a dense concentration of microspheres in the collagen gel. Injectability was determined by extrusion through 27 gauge needles. Samples were placed in 1.0 mL syringes for subsequent testing in the rabbit ear model.

### Example 8

### Rapidly Polymerizing Collagen Plus Derivatized Collagen Composition

### Preparation of Rapidly Polymerizing Collagen

Pure, soluble, porcine Type I collagen was purchased from SunMax Biotechnology, LTD. Saturated sodium chloride solution was added to the soluble, pepsin-digested collagen solution (3 mg/mL) to a concentration of 0.8M to precipitate collagen. The white, opaque precipitate was recovered by centrifugation for 15 minutes at 5,000 RPM. The concentrated collagen precipitate was placed in dialysis tubing with a molecular weight cut off of 10,000 daltons, or in dialysis cassette with a molecular weight cut-off of 20,000 daltons, and dialyzed against 0.5M acetic acid for at least 16-18 hours and then 0.1M acetic acid for at least 16-18 hours. The resulting clear, viscous, redissolved collagen concentrate was then dialyzed against 0.035M (35 mM) EDTA (ethylenediaminetetraacetic acid, disodium salt dihydate, SigmaUltra-99%). It was important to dialyze against disodium EDTA concentrations of at least 25 mM and preferably as high as 35 mM. The starting pH was 5.0±0.2. Dialysis was continued for at least 12 hours. The dialysis tubing or cassette was then transferred into a dialysis chamber containing 35 mM EDTA at a pH of 5.5±0.2 and dialyzed for at least 12 hours. The dialysis tubing or cassette was again transferred into a dialysis chamber containing 35 mM EDTA at a pH of 6.0 and dialyzed for at least 12 hours. Subsequent dialysis steps against 35 mM EDTA at pH 6.5 and 7.2 were conducted to bring the final collagen pH to approximately 7.0. The final clear, viscous collagen exhibited a pH of approximately 7.1 and did not undergo fibril formation at room temperature. Collagen fibrillogenesis was not triggered until the collagen was exposed to physiological liquids or liquids containing ions to trigger gelation and polymerizing reactions.

Rapidly polymerizing collagen (RPC) was supplemented with derivatized collagen (DC) to provide a mixture composition of 80% (v/v) rapidly polymerizing collagen and 20% derivatized collagen. The mixture was centrifuged and examined by injection into a neutral pH sodium phosphate buffer.

Following injection into the buffer, white fibrous units formed instantaneously. However, the fibrous units were thicker and less dense than the fibrous units formed by rapidly polymerizing collagen without the added derivatized collagen (FIG. 4). The combined collagen composition appears to provide additional volume for treating tissue depressions.

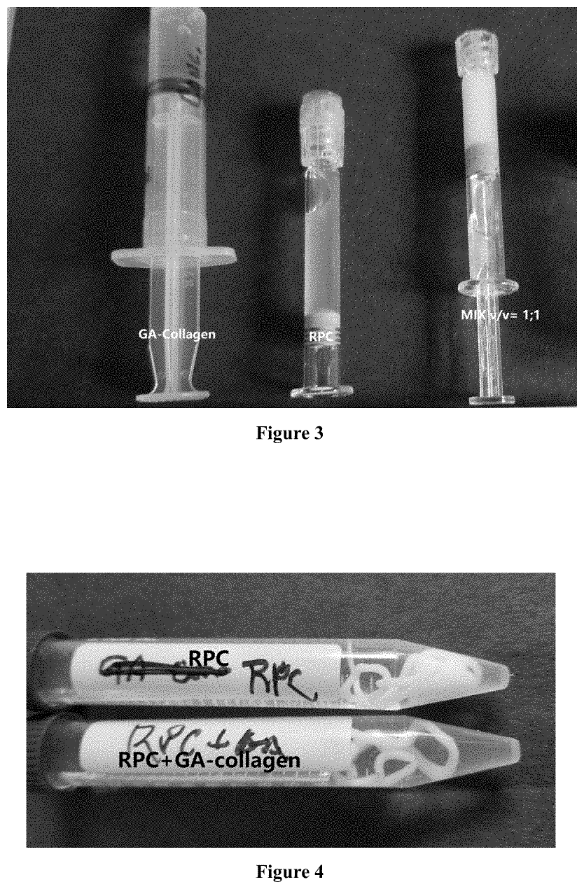

The longevity of the rapidly polymerizing collagen combined with the derivatized collagen was evaluated in subsequent animal evaluations. Different ratios of RPC and DC were studied. The results show that longevity of mixture depends on RPC rather than DC, however, addition of DC helps stabilizing un-polymerizing RPC before injection without affecting RPC polymerization ability in PBS and in vivo. (FIGS. 3 and 4)

### Example 9

### Derivatized Collagen Plus Crosslinked HA microspheres

Crosslinked HA microspheres were obtained through emulsified crosslinking reaction. 1˜3% by weight hyaluronic acid (MW>1500 kDa)in PBS solution and crosslinker ϵ-polylysine and 4-methylmorpholine hydrochloride (DMTMM) in 0.9% sodium chloride solution at the same mole ratio with HA were mixed and reacted in 5 times volume of organic oil silicone oil and Span 80 mixture (v:v=1.0˜2.0: 25) and stirred at 1,000 rpm to 1,500 rpm under 30 ˜45 ° C. for over 24 hours. Stirring in higher speed produced microspheres with smaller diameter. The additional organic oils are washed by n-hexane and ethyl acetate for 3 times and absolute ethanol for 3 times. The crosslinked HA microspheres were gathered through centrifugation at 8,000˜10,000 rpm for 10 min. After rehydration by PBS, crosslinked HA microsphere suspension was filtered to gather the right size (20˜50 μm) of microspheres. White powder of HA microspheres was obtained after ethanol precipitation and rehydration with neutral PBS.

The HA microspheres were homogeneously mixed (v:v=1:1) into the collagen gel (prepared in Example 1) using two syringes attached by a female-female syringe adaptor. Samples of the HA microspheres-collagen gel composition were examined microscopically showing a dense concentration of microspheres in the collagen gel. Injectability was determined by extrusion through 27 gauge needles. Samples were placed in 1.0 mL syringes for subsequent testing in the rabbit ear model.

### Example 10

### Concentrated Rapidly Polymerizing Collagen and PMMA Microsphere Mixture

RPC or derivatized collagen gel with high concentration of collagen usually have higher storage modulus which is higher than 1,200 Pa (5 Hz) with a very high dymamic viscosity. The large batch mixing of collagen gel and microsphere is a challenge. Vacuum planetary mixer with proper speed and time setting will easily mix the powder and viscous gel properly without introducing any air bubble.

PMMA microspheres (Phosphorex) with diameters from 32-50 μm were mixed into the RPC solution (prepared in Example 8) (collagen concention over 5%) at 20% by weight of the collagen gel. Composition was mixed by THINKY Mixer in a container with a cooler adapter at 1,000 rpm revolution speed (autorotation speed was half of the revolution speed) for 10 minutes under aseptic process for 3 times. A homogeneous cohesive composite without any bubble was obtained. Samples of the RPC-PMMA gel composition were examined microscopically showing a dense concentration of microspheres in the collagen gel. Injectability was determined by extrusion through 27 gauge (thin wall) needles. Samples were placed in 1.0 mL syringes for subsequent testing in the rabbit ear model.

0.2 mL of RPC-PMMA or Bellafill (Control; a commercial product available from SUNEVA Medical, which contains neutural bovine collagen and PMMA microspheres) were subcutaneously injected in rabbit ear (avoiding close to blood vessels). The rabbit ear thickness and the length, width and the thickness of bumps of implantation were measured before and right after injection (day 0), at day 1, day 7, day 14 and day 21. All measurements are taken by vernier caliper by the same person for three times and the mean values were calculated for further evaluation. The height and the estimate volume of implantation were calculated by the following equation:

Implantation height (mm)=thickness of ear and implantation−thickness of ear

Estimated volume of implantation (mm³)=⅙π×implantation height×implantation length×implantation width

Comparing to the commercial product Bellafill®, RPC with its unique unfibirillar state in neutrual pH allows a high concentrated collagen plus PMMA microspheres formulation extrudable with syringe needles.

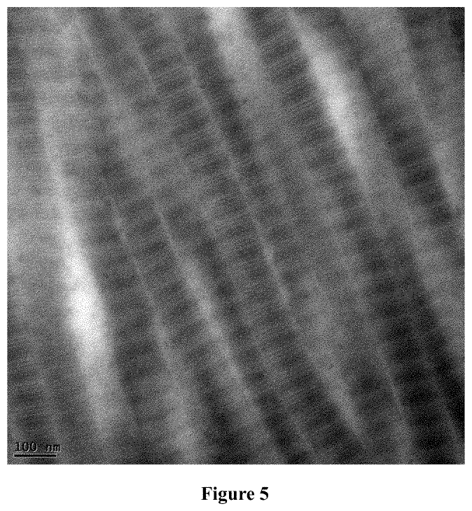

After injection, the RPC went polymerizing in situ in 5 minutes. (FIG. 5) RPC started fibrillogenesis form a gel with a increasing of storage modulus (up to 3,000 Pa under 5 Hz in vitro and 5,000˜15,000 Pa in vivo) And the implants formed a multidimentional scaffold highly similar to natural collagen scaffold in dermis.

The RPC collagen scaffold allowed cell immediate infiltration without any implant displacement. RPC has unexpectedly longer durability comparing to Bellafill, shortened or overcomed the yielding period of augmentation effect before collagen stimulation effect is awareable. (Table 1)

TABLE 1
Implantation morphology changes of RPC-PMMA and Bellafill
in 3 weeks in rabbit ear (the estimated volume of implant and heighth of
implantation were measured).

| Height of implantation (mm) | | | | | | Volume Estimate of implantation (mm³) | | | | | |
|---|---|---|---|---|---|---|---|---|---|---|---|
| Day | 0 | 1 | 7 | 14 | 21 | Day | 0 | 1 | 7 | 14 | 21 |

Rabbit #1

| | | | | | | | | | | | |
|---|---|---|---|---|---|---|---|---|---|---|---|
| RPC + PMMA | 3.79 | 2.33 | 2.93 | 2.46 | 2.22 | RPC + PMMA | 286.65 | 194.02 | 228.91 | 197.88 | 224.83 |
| Bellafill | 1.13 | 0.77 | 2.10 | 0.96 | 0.01 | Bellafill | 52.68 | 28.31 | 104.51 | 42.39 | 0.38 |

Rabbit #2

| | | | | | | | | | | | |
|---|---|---|---|---|---|---|---|---|---|---|---|
| RPC + PMMA | 3.12 | 3.14 | 2.20 | 1.88 | 2.21 | RPC + PMMA | 211.77 | 230.92 | 146.97 | 128.71 | 192.15 |
| Bellafill | 1.85 | 2.11 | 1.93 | 1.00 | 1.56 | Bellafill | 82.58 | 86.03 | 84.20 | 46.14 | 70.10 |

Later, neocollagen stimulated by the microspheres replaced the implantation, augmenting skin deficiency, finally after the slow degradation of collagen, PMMA with neocollagenesis can still maintain implantation and a natural augmentation effect.

### Example 11

### Concentrated Rapidly Polymerizing Collagen and Crosslinked HA Microsphere Composite

Crosslinked HA microspheres were obtained through emulsified crosslinking reaction (as described in Example 9). 1˜3% Crosslinker ϵ-polylysine and 4-methylmorpholine hydrochloride (DMTMM) 0.9% sodium chloride solution and 1˜3% hyaluronic acid (MW>1500 kDa) solution was mixed and reacting in 5 times volume of organic oils such as olive oil or silicone oil and spam 80 mixture (v:v=1.0˜2.0:25) and stirring at 1000 rpm to 1500 rpm under 30° C.˜45° C. for over 24 hours. The higher the speed is, the small diameter of microsphere will be obtained. The additional organic oils are washed by n-hexane and ethyl acetate for 3 times and absolute ethanol for 3 times and the crosslinked HA microspheres are gathered through centrifugation at 8000˜10000rpm for 10 min. After rehydration by PBS, crosslinked HA microsphere suspension was filtered to gather the right size of microspheres.

RPC gel with high concentration of collagen usually have higher storage modulus which is higher than 1,200Pa (5Hz) with a very high dymamic viscosity. The large batch mixing of collagen gel and microsphere is a challenge. Vacuum planetary mixer with proper speed and time setting will easily mix the powder and viscous gel properly without introducing any air bubble.

HA microspheres with diameters from 20-50 μm were mixed into the RPC solution (Example 8) (collagen concention over 5%) at 20% by weight of the collagen gel. Composition was mixed by THINKY Mixer in a container with a cooler adapter at 1,000 rpm revolution speed (autorotation speed was half of the revolution speed) for 10 minutes under aseptic process for 3 times. A homogeneous cohesive composite without any bubble was obtained. Samples of the HA-RPC gel composition were examined microscopically showing a dense concentration of microspheres in the collagen gel.

### OTHER EMBODIMENTS

Although the present invention has been described with reference to preferred embodiments, one skilled in the art can easily ascertain its essential characteristics and without departing from the spirit and scope thereof, can make various changes and modifications of the invention to adapt it to various usages and conditions. Those skilled in the art will recognize or be able to ascertain using no more than routine experimentation, many equivalents to the specific embodiments of the invention herein. Such equivalents are intended to be encompassed in the scope of the present invention.

All references, including patents, publications, and patent applications, mentioned in this specification are herein incorporated by reference in the same extent as if each independent publication, patent or patent application was specifically and individually indicated to be incorporated by reference.

### REFERENCES

### Publications:

Cockerham and Hsu (Facial Plastic Surgery, 25:106-113, 2009)
Denton and Shoman (Office Based Cosmetic Procedures & Technology, Section 3, pp 59-64, 2010).
Tagle et. al. (2010). J. February 5, 2010 J. Aesthetic and Clinical Dermatology

### Patents:

Miyata (U.S. Pat. No. 4,164,559)
DeVore, et al (U.S. Pat. Nos. 4,713,446, 4,851,513, 4,969,912, 5,067,961, 5,104,957, 5,201,764, 5,219,895, 5,332,809, 5,354,336, 5,476,515, 5,480,427, 5,631,243, 6,161,544 and 10,111,981)

We claim:

1. A composition for application in soft tissue augmentation comprising
(i) derivatized collagen solutions or rapidly polymerizing collagen gels (before undergoing fibrillogenesis) or crosslinked or uncrosslinked collagen fibrils; and
(ii) non-resorbable or slowly resorbable particles, spheres or granules contained in part (i).

2. The composition of claim 1, wherein the source of collagen for part (i) is selected from allogenetic, mammal hides or marine species or axolotl hides derived matrix; and/or
the collagen is selected from full collagen or atelocollagen, or recombinant collagen or recombinant collagen peptides from microorganism, plants, insect cells or animal cells, or collagen mimic peptides.

3. The composition of claim 1, wherein the derivatized collagen is derivatized with acetylation agents that alter the pKa of collagen and has one or more of the following features:
(a) soluble at neutral pH (such as 6.5-7.5);
(b) does not undergo fibrillogenesis at physiological pH; and/or (c) precipitates at acidic pH (such as 3.5-5.5, preferred 4.0˜5.0).

4. The composition of claim 1, wherein the derivatized collagen is derivatized with one or more agents selected from the group consisting of glutaric anhydride, succinic anhydride, maleic anhydride, citric acid anhydride, oxalic acid anhydride and ethylenediamine tetraacetic anhydride.

5. The composition of claim 1, wherein the rapidly polymerizing collagen gels are as described in U.S. Pat. No. 10,111,981B2; and/or
the rapidly polymerizing collagen gels comprises a neutralized solution comprising an acid soluble collagen, EDTA and a polyol, and wherein the acid soluble collagen comprises collagen selected from the group consisting of Type I collagen, Type III collagen and combinations thereof.

6. The composition of claim 5, wherein the acid soluble collagen in a concentration between 5 and 70 mg/ml; and/or
wherein said EDTA is disodium EDTA; and/or
wherein said EDTA is in a concentration between 10 and 50 mM; and/or
wherein said polyol is a sugar alcohol, such as D-mannitol; and/or
wherein said polyol is in a concentration between 2.5% and 4% (w/v); and/or
wherein said rapidly polymerizing collagen gels further comprises a disaccharide, fructose, or combinations thereof; and/or
wherein said rapidly polymerizing collagen gel has an osmolality of 280-360 mmol/kg.

7. The composition of claim 1, wherein the crosslinked collagen is crosslinked by one or more of the chemical agents selected from the group selected from: aldehyde,such as methyl aldehyde, oxalaldehyde, glutaraldehyde and butenoic aldehyde; or iridoids such as genipin; or carbodiimide such as dicyclohexyl carbodiimide; or epoxide such as 1,4-butanediol diglycidyl ether (BDDGE), or acyl azide; or saccharides such as ribose or glucose.

8. The composition of claim 1, wherein the non-resorbable or slowly resorbable particles, spheres or granules are one or more selected from the group consisting of:
reconstituted or crosslinked collagen fibrils;
Polymethylmethacrylate (PMMA) microspheres;
polymethylmethacrylate-hydroxyapatite microspheres;
crosslinked hyaluronic acid microspheres produced by emulsified crosslinking reaction, double emulsion evaporation method, microfluidic crosslinking reaction, or stamp formation;
polyethylane glyco (PEG) microspheres;
PEG-hydroxyapatite microspheres;
poly-L-lactide (PLA) microspheres;
PEG-PLA copolymer microspheres;
poly-L-lactide-hydroxyapatite microspheres;
polyglycolic acid (PGA) microspheres;
poly-L-lactide-hydroxyapatite microspheres;
polylactide and polyglycolide polymers and copolymers (PLGA) microspheres;
poly ϵ-caprolactone (PCL) microspheres;
PCL-PLA copolymer microspheres;
poly-ϵ-caprolactone-hydroxyapatite microspheres;
poly(p-dioxanone) (PDO) microspheres;
poly(p-dioxanone)-hydroxyapatite microspheres;
calcium hydroxyapatite; and
L-Lactide/Trimethylene Carbonate Copolymer granules.

9. The composition of claim 8, wherein the crosslinker of hyaluronic acid micrspheres is selected from divinylsulfone, glutaraldehyde, 1,4-butanediol diglycidyl ether, p-phenylene biscarbodiimide, 1,2,7,8-diepoxyoctane or oligomers rich in amino groups (such as poly-lysine or poly-arginine or γ-polyglutamic acid).

10. The composition of claim 8, wherein the crosslinked hyaluronic acid microspheres is coated with bio-degradable polymers, such as poly-L-lactide (PLA), polyethylene glycol (PEG), or PLGA, or poly(p-dioxanone) (PDO).

11. The composition of claim 1, wherein the size of the non-resorbable or slowly resorbable particles, spheres or granules is ranged from 5 to 150 μm, preferably from 20 to 50 μm; and/or
wherein the non-resorbable or slowly resorbable particles, spheres or granules are obtained through spray-precipitation technique, emulsion, double emulsion evaporation method, microfluidic reaction, Solid-Gel process, melt extrusion technique, sintering process or stamp formation; and/or
wherein the non-resorbable or slowly resorbable particles, spheres or granules are sterilized through heat moist sterilization, gamma irradiation or ethylene oxide sterilization.

12. The composition of claim 1, wherein the amount of collagen in part (i) is from 0.1 wt % to 10 wt %, and the amount of part (ii) is from 1 wt % to 55wt %, based on the total weight of the composition.

13. The composition of claim 1, further comprising additive(s) in an amount of from 0 to 5 wt %, based on the total weight of the composition.

14. The composition of claim 1, wherein the additive is selected from the group consisting of
local anesthesia drugs such as lidocaine, procaine, preferably in a concentration of from 0.1% to 0.5% by weight; and/or
polyols stabilizers, such as glycerin, mannitol, butanediol, sorbitol, preferably in a concentration of from 0.1 to 5% weight; and/or
a stabilizer with chelating ability, such as EDTA, EGTA, citric acid, sodium citrate, preferably in a concentration of from 0.1 to 5% by weight; and/or
a sulfur stablizer or dissolution promotor, such as Chondroitin Sulfate Sodium (CS), Gluscosamine Sulphate (GS) or Methyl sulfonyl methane (MSM), preferably in a concentration of from 0.1% to 5% by weight; and/or
soluble small molecules added through dialysis process, ultrafitration or tangential flow ultrafiltration with organic membranes or ceramic membrane with MWCO>10 KDa.

15. The composition of claim 1, wherein part (ii) is added to part (i) by utilizing vacuum planetary mixer to form an injectable homogeneous gel, preferably with a revolution speed of 200 rpm˜1,400 rpm and an autorotation speed of 100 rpm˜700 rpm, preferably with a mixing time of 10˜30 minutes with vacuum.

16. The composition of claim 1, wherein part (ii) is added to a salt or salt or pH precipitate of part (i) and re-solublized by dialysis or ultradialysis or ultrafiltration process to form a homogeneous injectable gel.

17. A method for the preparation of the composition of claim 1, comprising:
combining part (i) with part (ii), for example by
adding part (ii) to part (i) by utilizing vacuum planetary mixer to form an injectable homogeneous gel, preferably with a revolution speed of 200 rpm˜1,400 rpm and an autorotation speed of 100 rpm˜700 rpm, preferably with a mixing time of 10˜30 minutes with vacuum; and/or
adding part (ii) to a salt or salt or pH precipitate of part (i) and re-solublized by dialysis or ultradialysis or ultrafiltration process to form a homogeneous injectable gel.

18. A method for augmenting soft tissue in a subject in need thereof, comprising injecting the composition of claim 1 to the site in need of the augment.

19. The method of claim 18, wherein the composition is injected into soft tissue to correct soft tissue deficiencies; and/or
wherein the composition is injected into dermis to correct soft tissue deficiencies including wrinkles, dermal folds, dermal laxity, unevenness, facial emaciation, fat atrophy, cheek depression, eye socket depression, or a combination thereof; and/or
wherein the composition is injected into tissues other than dermis, including cartilage, to correct tissue deficiencies.

20. The method of claim 18 wherein the composition is injectable through a 25˜30 gauge needle or cannula, such as a 25, 27 or 30 gauge needle or cannula.
